(19) United States
(12) Patent Application Publication
(10) Pub. No.: US 2026/0364568 A1
(43) Pub. Date: Nov. 19, 2026

(54) METHOD FOR THE PRODUCTION OF COLOUR-STABLE SUPERABSORBER PARTICLES

(71) Applicant: BASF SE, Ludwigshafen Am Rhein (DE)

(72) Inventors: Christian HILS, Ludwigshafen am Rhein (DE); Christophe BAUDUIN, Ludwigshafen am Rhein (DE); Jan Niclas GORGES, Ludwigshafen am Rhein (DE); Matthias WEISMANTEL, Ludwigshafen am Rhein (DE); Ruediger FUNK, Niedernhausen (DE)

(73) Assignee: BASF SE, Ludwigshafen Am Rhein (DE)

(21) Appl. No.: 19/133,781

(22) PCT Filed: Nov. 20, 2023
(86) PCT No.: PCT/EP2023/082340

§ 371 Date: May 29, 2025

(30) Foreign Application Priority Data
Nov. 29, 2022 (EP) 22210280.8

(51) Int. Cl. B01J 20/26; A61L 15/24; A61L 15/60; B01J 20/30; B01J 20/32; C08F 20/06; C08J 3/24

(52) U.S. Cl. CPC B01J 20/267; A61L 15/24; A61L 15/60; B01J 20/3085; B01J 20/321; B01J 20/3217; B01J 20/3255; C08F 20/06; C08J 3/24

(57) ABSTRACT

The present invention relates to a process for producing color-stable superabsorbent particles, wherein an aqueous monomer solution or suspension is polymerized to a polymer gel, the resultant polymer gel is optionally comminuted, the polymer gel is then dried, the dried polymer gel is optionally ground and classified, then the dried polymer gel is thermally surface postcrosslinked and cooled, which comprises coating with a pyrazole after the polymerization.

The present invention relates to a process for producing color-stable superabsorbent particles, wherein an aqueous monomer solution or suspension is polymerized to a polymer gel, the resultant polymer gel is optionally comminuted, the polymer gel is then dried, the dried polymer gel is optionally ground and classified, then the dried polymer gel is thermally surface postcrosslinked and cooled, which comprises coating with a pyrazole after the polymerization.

Superabsorbents are used to produce diapers, tampons, sanitary napkins and other hygiene articles, but also as water-retaining agents in market gardening. Superabsorbents are also referred to as water-absorbing polymers.

The production of superabsorbents is described in the monograph “Modern Superabsorbent Polymer Technology”, F. L. Buchholz and A. T. Graham, Wiley-VCH, 1998, pages 71 to 103.

To improve the performance properties, for example gel bed permeability (GBP) and absorption under a pressure of 49.2 g/cm² (AUL0.7 psi), superabsorbent particles are generally surface postcrosslinked. This increases the level of crosslinking of the particle surface, which can at least partly decouple the absorption under a pressure of 49.2 g/cm² (AUL0.7 psi) and the centrifuge retention capacity (CRC). This surface postcrosslinking can be performed in the aqueous gel phase. Preferably, however, polymer particles (base polymer), having been dried, ground and sieved off, are surface coated with a surface postcrosslinker and thermally surface postcrosslinked. Crosslinkers suitable for that purpose are compounds which can form covalent bonds with at least two carboxylate groups of the polymer particles.

WO 2021/105038 A1 and the prior PCT application with reference PCT/EP2022/059572 disclose pyrazoles as polymerization inhibitors.

It was an object of the present invention to provide an improved process for producing color-stable superabsorbent particles.

The object was achieved by a process for producing surface postcrosslinked superabsorbent particles by polymerizing an aqueous monomer solution or suspension comprising
a) at least one ethylenically unsaturated monomer which bears acid groups and is at least partly neutralized,
b) at least one crosslinker and
c) at least one initiator,
wherein the aqueous monomer solution or suspension is polymerized to a polymer gel, the resultant polymer gel is optionally comminuted, the polymer gel is then dried, the dried polymer gel is optionally ground and classified, then the dried polymer gel is thermally surface postcrosslinked and cooled, which comprises coating the resultant polymer particles with at least one pyrazole before, during or after the thermal surface postcrosslinking.

The pyrazole is typically a monomeric pyrazole.

The pyrazole used in the process of the invention is preferably a compound of the general formula (I)

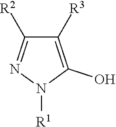

where
R¹ is C₁- or C₂-alkyl,
R² is H, C₁- to C₂₀-alkyl or C₆- to C₂₀-arylalkyl and
R³ is H, C₁- to C₂₀-alkyl or C₆- to C₂₀-arylalkyl,
or a compound of the general formula (II)

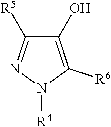

where
R⁴ is C₁- or C₂-alkyl,
R⁵ is H, C₁- to C₂₀-alkyl or C₆- to C₂₀-arylalkyl and
R⁶ is H, C₁- to C₂₀-alkyl or C₆- to C₂₀-arylalkyl,
or a compound of the general formula (III)

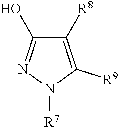

where
R⁷ is C₁- or C₂-alkyl,
R⁸ is H, C₁- to C₂₀-alkyl or C₆- to C₂₀-arylalkyl and
R⁹ is H, C₁- to C₂₀-alkyl or C₆- to C₂₀-arylalkyl.

The alkyl groups may be linear, branched and/or cyclic.

The pyrazole used in the process of the invention is more preferably a compound of the general formula (I)

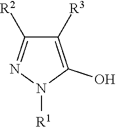

where
R¹ is C₁-alkyl,
R² is C₁- to C₃-alkyl or C₆- to C₈-arylalkyl and
R³ is H, C₁- to C₃-alkyl or C₆- to C₈-arylalkyl,
or a compound of the general formula (II)

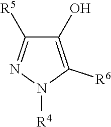

where
R⁴ is C₁-alkyl,
R⁵ is C₁- to C₃-alkyl or C₆- to C₈-arylalkyl and
R⁶ is H, C₁- to C₃-alkyl or C₆- to C₈-arylalkyl,
or a compound of the general formula (III)

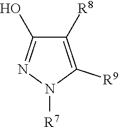

where
R⁷ is C₁-alkyl,
R⁸ is H, C₁- to C₃-alkyl or C₆- to C₈-arylalkyl and
R⁹ is H, C₁- to C₃-alkyl or C₆- to C₈-arylalkyl.

The alkyl groups may be linear, branched and/or cyclic.

The compounds of the general formula (I) are in equilibrium with their keto form. For example, 1,3-dimethyl-5-pyrazolone is the keto form of 1,3-dimethyl-5-hydroxypyrazole.

The pyrazole used in the process of the invention is most preferably 1,3-dimethyl-5-pyrazolone, 1,5-dimethyl-3-ethyl-4-hydroxypyrazole or 1,5-dimethyl-4-hydroxy-3-phenylpyrazole.

The polymer particles are coated with preferably 0.001% to 1% by weight, more preferably 0.005% to 0.2% by weight, most preferably 0.01% to 0.1% by weight, of the pyrazole, based in each case on the polymer particles.

The present invention is based on the finding that pyrazoles distinctly improve the color stability of superabsorbents.

Acrylic acid is the preferred ethylenically unsaturated carboxylic acid. Peroxodisulfate, especially ammonium peroxodisulfate, sodium peroxodisulfate and/or potassium peroxodisulfate, is the preferred initiator c).

The production of the superabsorbents is described in detail hereinafter:

The superabsorbents are produced by polymerizing a monomer solution and are typically water-insoluble.

The ethylenically unsaturated monomers a) bearing acid groups are preferably water-soluble, i.e. their solubility in water at 23 °C is typically at least 1 g/100 g of water, preferably at least 5 g/100 g of water, more preferably at least 25 g/100 g of water and most preferably at least 35 g/100 g of water.

Suitable monomers are, for example, ethylenically unsaturated carboxylic acids, such as acrylic acid, methacrylic acid and itaconic acid. Particularly preferred monomers are acrylic acid and methacrylic acid. Very particular preference is given to acrylic acid.

The ethylenically unsaturated monomers a) bearing acid groups have typically been partly neutralized. The neutralization is conducted at the monomer stage. This is typically accomplished by mixing in the neutralizing agent as an aqueous solution or else preferably as a solid. The degree of neutralization is preferably from 40 to 85 mol %, more preferably from 50 to 80 mol % and most preferably from 60 to 75 mol %, for which the customary neutralizing agents can be used, preferably alkali metal hydroxides, alkali metal oxides, alkali metal carbonates or alkali metal hydrogencarbonates and also mixtures thereof. Instead of alkali metal salts, it is also possible to use ammonium salts. Particularly preferred alkali metals are sodium and potassium, but very particular preference is given to sodium hydroxide, sodium carbonate or sodium hydrogencarbonate and also mixtures thereof, especially sodium hydroxide.

The monomers typically comprise polymerization inhibitors, preferably hydroquinone monoethers, as storage stabilizers.

Suitable crosslinkers b) are compounds having at least two groups suitable for crosslinking. Such groups are, for example, ethylenically unsaturated groups which can be polymerized free-radically into the polymer chain, and functional groups which can form covalent bonds with the acid groups of the monomer. In addition, polyvalent metal salts which can form coordinate bonds with at least two acid groups of the monomer are also suitable as crosslinkers.

Suitable crosslinkers b) are, for example, ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane, as described in EP 0 530 438 A1, di- and triacrylates, as described in EP 0 547 847 A1, EP 0 559 476 A1, EP 0 632 068 A1, WO 93/21237 A1, WO 03/104299 A1, WO 03/104300 A1, WO 03/104301 A1 and DE 103 31 450 A1, mixed acrylates which, as well as acrylate groups, comprise further ethylenically unsaturated groups, as described in DE 103 31 456 A1 and DE 103 55 401 A1, or crosslinker mixtures, as described, for example, in DE 195 43 368 A1, DE 196 46 484 A1, WO 90/15830 A1 and WO 02/032962 A2.

The amount of crosslinker b) is preferably 0.05% to 1.5% by weight, more preferably 0.1% to 1% by weight and most preferably 0.15% to 0.6% by weight, calculated in each case on the basis of the total amount of monomer used. With rising crosslinker content, the centrifuge retention capacity (CRC) falls and the absorption under a pressure of 21.0 g/cm² (AUL0.3 psi) passes through a maximum.

Initiators c) used may be all compounds which generate free radicals under the polymerization conditions, for example thermal initiators, redox initiators or photoinitiators. Suitable redox initiators are sodium peroxodisulfate/ascorbic acid, hydrogen peroxide/ascorbic acid, sodium peroxodisulfate/sodium bisulfite and hydrogen peroxide/sodium bisulfite. Preference is given to using mixtures of thermal initiators and redox initiators, such as sodium peroxodisulfate/hydrogen peroxide/ascorbic acid. The reducing component used is preferably the disodium salt of 2-hydroxy-2-sulfonatoacetic acid or a mixture of the sodium salt of 2-hydroxy-2-sulfinatoacetic acid, the disodium salt of 2-hydroxy-2-sulfonatoacetic acid and sodium bisulfite. Such mixtures are obtainable as Bruggolite® FF6 and Brüggolite® FF7 (Brüggemann Chemicals; Heilbronn; Germany).

The water content of the monomer solution is preferably from 40% to 75% by weight, more preferably from 45% to 70% by weight and most preferably from 50% to 65% by weight. As the water content rises, the energy expenditure in the subsequent drying rises and, as the water content falls, the heat of polymerization can only be removed inadequately.

The temperature of the monomer solution is preferably from 10 to 90 °C, particularly preferably from 20 to 70 °C, very particularly preferably from 30 to 50 °C.

For optimal action, the preferred polymerization inhibitors require dissolved oxygen. The monomer solution can therefore be freed of dissolved oxygen before the polymerization by inertization, i.e. flowing an inert gas through, preferably nitrogen or carbon dioxide. The oxygen content of the monomer solution is preferably lowered before the polymerization to less than 1 ppm by weight, more preferably to less than 0.5 ppm by weight, most preferably to less than 0.1 ppm by weight.

Suitable reactors for the polymerization are, for example, kneading reactors or belt reactors. In the kneader, the polymer gel formed in the polymerization of an aqueous monomer solution or suspension is comminuted continuously by, for example, contrarotatory stirrer shafts, as described in WO 2001/038402 A1. Polymerization on the belt is described, for example, in DE 38 25 366 A1 and U.S. Pat. No. 6,241,928. Polymerization in a belt reactor forms a polymer gel which has to be comminuted, for example in an extruder or kneader.

To improve the drying properties, the comminuted polymer gel obtained by means of a kneader can additionally be extruded.

The polymer gel is then typically dried with an air circulation belt drier until the residual moisture content is preferably 0.5 to 10% by weight, more preferably 1 to 7% by weight and most preferably 2 to 5% by weight, the residual moisture content being determined by EDANA recommended test method No. WSP 230.2-05 “Mass Loss Upon Heating”. In the case of too high a residual moisture content, the dried polymer gel has too low a glass transition temperature T_{g} and can be processed further only with difficulty. In the case of too low a residual moisture content, the dried polymer gel is too brittle and, in the subsequent comminution steps, undesirably large amounts of polymer particles with an excessively low particle size are obtained (“fines”). The solids content of the polymer gel before the drying is preferably from 25% to 90% by weight, more preferably from 35% to 70% by weight, most preferably from 40% to 60% by weight. Subsequently, the dried polymer gel is crushed and optionally coarsely comminuted.

Thereafter, the dried polymer gel is typically ground and classified, and the apparatus used for grinding may typically be single- or multistage roll mills, preferably two- or three-stage roll mills, pin mills, hammer mills or vibratory mills.

The average particle size of the polymer particles removed as the product fraction is preferably from 150 to 850 μm, more preferably from 250 to 600 μm, very particularly from 300 to 500 μm. The average particle size of the product fraction may be determined by means of EDANA recommended test method No. WSP 220.2 (05) “Particle Size Distribution”, where the proportions by mass of the screen fractions are plotted in cumulative form and the average particle size is determined graphically. The average particle size here is the value of the mesh size which arises for a cumulative 50% by weight.

To further improve the properties, the polymer particles are thermally surface postcrosslinked. Suitable surface postcrosslinkers are compounds which comprise groups which can form covalent bonds with at least two carboxylate groups of the polymer particles. Suitable compounds are, for example, polyfunctional amines, polyfunctional amido amines, polyfunctional epoxides, as described in EP 0 083 022 A2, EP 0 543 303 A1 and EP 0 937 736 A2, di- or polyfunctional alcohols, as described in DE 33 14 019 A1, DE 35 23 617 A1 and EP 0 450 922 A2, or β-hydroxyalkylamides, as described in DE 102 04 938 A1 and U.S. Pat. No. 6,239,230.

The amount of surface postcrosslinker is preferably 0.001% to 2% by weight, more preferably 0.01% to 1% by weight and most preferably 0.03% to 0.7% by weight, based in each case on the polymer particles.

In a preferred embodiment of the present invention, polyvalent cations are applied to the particle surface in addition to the surface postcrosslinkers.

The polyvalent cations usable in the process of the invention are, for example, divalent cations such as the cations of zinc, magnesium, calcium and strontium, trivalent cations such as the cations of aluminum, iron, chromium, rare earths and manganese, tetravalent cations such as the cations of titanium and zirconium. Possible counterions are chloride, bromide, hydroxide, sulfate, hydrogensulfate, carbonate, hydrogencarbonate, nitrate, phosphate, hydrogenphosphate, dihydrogenphosphate and carboxylate, such as acetate and lactate. Aluminum hydroxide, aluminum sulfate and aluminum lactate are preferred.

The amount of polyvalent cation used is, for example, 0.001% to 1.5% by weight, preferably 0.005% to 1% by weight and more preferably 0.02% to 0.8% by weight, based in each case on the polymer.

The surface postcrosslinking is typically performed in such a way that a solution of the surface postcrosslinker is sprayed onto the dried polymer particles. After the spray application, the surface postcrosslinker-coated polymer particles are subjected to thermal treatment.

The spray application of a solution of the surface postcrosslinker is preferably performed in mixers with moving mixing tools, such as screw mixers, disk mixers and paddle mixers. Particular preference is given to horizontal mixers such as paddle mixers, very particular preference to vertical mixers. The distinction between horizontal mixers and vertical mixers is made by the position of the mixing shaft, i.e. horizontal mixers have a horizontally mounted mixing shaft and vertical mixers have a vertically mounted mixing shaft. Suitable mixers are, for example, horizontal Pflugschar® plowshare mixers (Gebr. Lodige Maschinenbau GmbH; Paderborn; Germany), Vrieco-Nauta continuous mixers (Hosokawa Micron BV; Doetinchem; the Netherlands), Processall Mixmill mixers (Processall Incorporated; Cincinnati; USA) and Schugi Flexomix® (Hosokawa Micron BV; Doetinchem; the Netherlands). However, it is also possible to spray on the surface postcrosslinker solution in a fluidized bed.

The surface postcrosslinkers are typically used in the form of an aqueous solution. The penetration depth of the surface postcrosslinker into the polymer particles can be adjusted via the content of nonaqueous solvent and total amount of solvent.

The thermal treatment is preferably conducted in contact driers, more preferably paddle driers, most preferably disk driers. Suitable driers are, for example, Hosokawa Bepex® Horizontal Paddle Dryer (Hosokawa Micron GmbH; Leingarten; Germany), Hosokawa Bepex® Disk Dryer (Hosokawa Micron GmbH; Leingarten; Germany), Holo-Flite® driers (Metso Minerals Industries Inc.; Danville; USA) and Nara Paddle Dryer (NARA Machinery Europe; Frechen; Germany). Moreover, fluidized bed driers may also be used.

The surface postcrosslinking can be effected in the mixer itself, by heating the jacket or blowing in warm air. Equally suitable is a downstream drier, for example a tray drier, a rotary tube oven or a heatable screw. It is particularly advantageous to effect mixing and thermal surface postcrosslinking in a fluidized bed drier.

Preferred reaction temperatures are in the range of 100 to 250 °C, preferably 110 to 220 °C, more preferably 120 to 210 °C, most preferably 130 to 200 °C. The preferred dwell time at this temperature is preferably at least 10 minutes, more preferably at least 20 minutes, most preferably at least 30 minutes, and typically at most 60 minutes.

Subsequently, the surface postcrosslinked polymer particles can be classified again, with excessively small and/or excessively large polymer particles being removed and recycled into the process.

To further improve the properties, the surface postcrosslinked polymer particles can be coated or remoisturized.

The remoisturizing is preferably performed at 30 to 80 °C, more preferably at 35 to 70 °C, most preferably at 40 to 60 °C. At excessively low temperatures the polymer particles tend to form lumps, and at higher temperatures water already evaporates to a noticeable degree. The amount of water used for remoisturizing is preferably from 1% to 10% by weight, more preferably from 2% to 8% by weight and most preferably from 3% to 5% by weight. The remoisturizing increases the mechanical stability of the polymer particles and reduces their tendency to static charging. The remoisturizing is advantageously performed in a cooler after the thermal surface postcrosslinking.

Suitable coatings for improving the swell rate and the gel bed permeability (GBP) are, for example, inorganic inert substances, such as water-insoluble metal salts, organic polymers, cationic polymers and di- or polyvalent metal cations. Suitable coatings for dust binding are, for example, polyols. Suitable coatings for counteracting the undesired caking tendency of the polymer particles are, for example, fumed silica, such as Aerosil® 200, precipitated silica, such as Sipernat® D17, and surfactants, such as Span® 20.

The present invention further provides superabsorbent particles coated with at least one pyrazole.

The pyrazole is typically a monomeric pyrazole.

The pyrazole is preferably a compound of the general formula (I)

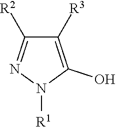

where
R¹ is C₁- or C₂-alkyl,
R² is H, C₁- to C₂₀-alkyl or C₆- to C₂₀-arylalkyl and
R³ is H, C₁- to C₂₀-alkyl or C₆- to C₂₀-arylalkyl,
or a compound of the general formula (II)

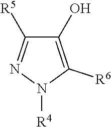

where
R⁴ is C₁- or C₂-alkyl,
R⁵ is H, C₁- to C₂₀-alkyl or C₆- to C₂₀-arylalkyl and
R⁶ is H, C₁- to C₂₀-alkyl or C₆- to C₂₀-arylalkyl,
or a compound of the general formula (III)

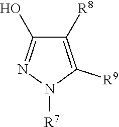

where
R⁷ is C₁- or C₂-alkyl,
R⁸ is H, C₁- to C₂₀-alkyl or C₆- to C₂₀-arylalkyl and
R⁹ is H, C₁- to C₂₀-alkyl or C₆- to C₂₀-arylalkyl.

The pyrazole is more preferably a compound of the general formula (I)

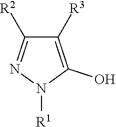

where
R¹ is C₁-alkyl,
R² is C₁- to C₃-alkyl or C₆- to C₈-arylalkyl and
R³ is H, C₁- to C₃-alkyl or C₆- to C₈-arylalkyl,
or a compound of the general formula (II)

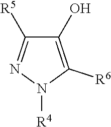

where
R⁴ is C₁-alkyl,
R⁵ is C₁- to C₃-alkyl or C₆- to C₈-arylalkyl and
R⁶ is H, C₁- to C₃-alkyl or C₆- to C₈-arylalkyl,
or a compound of the general formula (III)

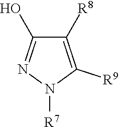

where
R⁷ is C₁-alkyl,
R⁸ is C₁- to C₃-alkyl or C₆- to C₈-arylalkyl and
R⁹ is H, C₁- to C₃-alkyl or C₆- to C₈-arylalkyl.

The alkyl groups may be linear, branched and/or cyclic.

The compounds of the general formula (I) are in equilibrium with their keto form. For example, 1,3-dimethyl-5-pyrazolone is the keto form of 1,3-dimethyl-5-hydroxypyrazole.

The pyrazole is most preferably 1,3-dimethyl-5-pyrazolone, 1,5-dimethyl-3-ethyl-4-hydroxypyrazole or 1,5-dimethyl-4-hydroxy-3-phenylpyrazole.

The superabsorbent particles have been coated with preferably 0.001% to 1% by weight, more preferably 0.005% to 0.2% by weight, most preferably 0.01% to 0.1% by weight, of the pyrazole, based in each case on the polymer particles.

Acrylic acid is the preferred ethylenically unsaturated carboxylic acid. Peroxodisulfate, especially ammonium peroxodisulfate, sodium peroxodisulfate and/or potassium peroxodisulfate, is the preferred initiator c).

The present invention further provides hygiene articles comprising superabsorbent particles of the invention.

### Methods

The measurements should, unless stated otherwise, be conducted at an ambient temperature of 23±2 °C and a relative air humidity of 50%+10%. The superabsorbent particles are mixed thoroughly before the measurement.

Color Value (CIE Color Numbers [L, a, b])

Color value is measured using a “LabScan XE Spectrometer” colorimeter (HunterLab; Reston; USA) by the CIELAB method (Hunterlab, volume 8, 1996, edition 7, pages 1 to 4). Colors are described via the coordinates L, a and b of a three-dimensional system. L characterizes brightness, where L=0 is black and L=100 white. The values for a and b respectively describe the color position on the red/green and yellow/blue color axes, where positive a values represent red colors, negative a values represent green colors, positive b values represent yellow colors, and negative b values represent blue colours.

The Hunter 60 value (HC60) is a measure of the whiteness of surfaces and is defined as L-3b, meaning that the lower the value, the darker and more yellow the color.

The test was conducted with a tissue culture dish (diameter 35 mm and height 10 mm) and a port plate opening of 0.5 inch.

Color index is measured in accordance with the tristimulus method according to DIN 5033-6.

### Yellowness Index (YI)

Yellowness index (YI) is measured to ASTM D1925 or to ASTM E313. The higher the value, the darker and more yellow the color.

### EXAMPLES

### Example 1

By continuously mixing deionized water, 50% by weight sodium hydroxide solution and acrylic acid, a monomer solution was prepared such that the degree of neutralization corresponded to 74.0 mol %. The water content of the monomer solution was 59.0% by weight.

The crosslinker used was 3-tuply ethoxylated glyceryl triacrylate (purity about 85% by weight). The amount used was 1.34 kg per t of monomer solution.

The free-radical polymerization was initiated using, per t of monomer solution, 2.14 kg of a 0.25% by weight aqueous hydrogen peroxide solution, 2.91 kg of a 15% by weight aqueous sodium peroxodisulfate solution and 1.97 kg of a 1% by weight aqueous ascorbic acid solution.

The monomer solution was introduced into a List Contikneter continuous kneader reactor with a capacity of 6.3 m³ (LIST AG, Arisdorf, Switzerland). The throughput of the monomer solution was about 20 t/h. The reaction solution had a feed temperature of 23.5 °C.

Between the addition point for the crosslinker and the addition sites for the hydrogen peroxide and sodium peroxodisulfate solutions, the monomer solution was inertized with nitrogen. Ascorbic acid was metered directly into the reactor.

After about 50% of the dwell time, an additional about 1000 kg/h of polymer particles obtained in the production process by comminution and classification, having a particle size of less than 150 μm, was metered into the reactor. The dwell time of the reaction mixture in the reactor was about 15 minutes.

The polymer gel obtained was applied to the conveyor belt of an air circulation belt drier by means of an oscillating conveyor belt. The air circulation belt drier had a length of 48 m. The conveyor belt of the air circulation belt drier had an effective width of 4.4 m. On the air circulation belt drier, an air/gas mixture (about 175 °C) flowed continuously around the aqueous polymer gel and dried it. The dwell time in the air circulation belt drier was 37 minutes.

The dried polymer gel was comminuted by means of a three-stage roll mill and sieved off to a particle size of 150 to 700 μm. Polymer particles having a particle size of less than 150 μm were separated off. Polymer particles having a particle size of greater than 700 μm were recycled into the comminution. Polymer particles having a particle size in the range from 150 to 700 μm were thermally surface postcrosslinked.

The polymer particles were coated with a surface postcrosslinker solution in a Schugi Flexomix® (Hosokawa Micron B.V., Doetinchem, the Netherlands) and then dried in a NARA Paddle Dryer (GMF Gouda, Waddinxveen, the Netherlands) at 175 °C for 45 minutes.

The following amounts were metered into the Schugi Flexomix®:
7.5 t/h of polymer particles
334.50 kg/h of surface postcrosslinker solution

The surface postcrosslinker solution comprised 1.35% by weight of ethylene glycol diglycidyl ether, 44.84% by weight of propane-1,2-diol and 53.81% by weight of water.

After drying, the surface postcrosslinked polymer particles were cooled down to about 60 °C in a NARA Paddle-Cooler (GMF Gouda, Waddinxveen, the Netherlands). At the same time, the surface postcrosslinked polymer particles were coated with 7.5 kg/h of a 50% by weight aqueous polyethylene glycol solution (polyethylene glycol having an average molar mass of 400 g/mol), 375 kg/h of water, 22.5 kg/h of aluminum trihydroxide (“dry aluminum hydroxide gel”, article number 511066100, Dr. Paul Lohmann GmbH KG, Hauptstrasse 2, 31860 Emmerthal, Germany) and 18.75 kg/h of a 1% by weight aqueous solution of sorbitan monolaurate.

### Example 2

20 g in each case of superabsorbent from example 1 was mixed with 200 ppm by weight in each case of a pyrazole and stored in a climate-controlled cabinet at 70 °C and 80% relative humidity for 14 days.

The results are collated in table 1.

| Pyrazole added | L | a | b | HC 60 | YI D1925 | YI E313 |
|---|---|---|---|---|---|---|
| none*) | 44.4 | 11.1 | 17.8 | −9.1 | 89.1 | 89.3 |
| 3-carboxypyrazole | 47.8 | 10.9 | 17.9 | −6.0 | 83.3 | 83.5 |
| 3,5-dimethyl-1-hydroxymethylpyrazole | 46.1 | 10.9 | 17.6 | −6.8 | 85.1 | 85.3 |
| 1,3-dimethyl-5-pyrazolone | 72.7 | 5.5 | 14.9 | 28.0 | 42.0 | 42.1 |
| 3-acetyl-1,5-dimethyl-4-hydroxypyrazole | 45.7 | 10.7 | 17.6 | −7.0 | 85.4 | 85.5 |
| 3-ethyl-4-hydroxy-1-isopropyl-5- | 48.3 | 10.7 | 18.1 | −5.9 | 82.6 | 82.8 |
| methylpyrazole | | | | | | |
| 3-hydroxy-1-methylpyrazole | 49.5 | 10.7 | 18.0 | −4.3 | 80.1 | 80.3 |
| 1,5-dimethyl-4-hydroxy-3-phenylpyrazole | 78.7 | 3.2 | 12.2 | 42.1 | 30.6 | 30.7 |
| 1,5-dimethyl-3-ethyl-4-hydroxypyrazole | 82.7 | 3.2 | 10.3 | 51.9 | 24.9 | 25.1 |

*)comparative example

### Example 3

20 g in each case of superabsorbent from example 1 was mixed with 1,5-dimethyl-3-ethyl-4-hydroxypyrazole and stored in a climate-controlled cabinet at 70 °C and 80% relative humidity for 14 days.

The results are collated in table 2.

| Amount added | L | a | b | HC 60 | YI D1925 | YI E313 |
|---|---|---|---|---|---|---|
| none*) | 44.9 | 10.9 | 16.8 | −5.6 | 84.2 | 84.4 |
| 50 ppm by wt. | 56.7 | 10.1 | 18.5 | 1.2 | 71.0 | 71.1 |
| 100 ppm by wt. | 73.0 | 6.8 | 14.7 | 29.0 | 42.5 | 42.7 |
| 150 ppm by wt. | 82.7 | 3.2 | 10.3 | 51.9 | 24.9 | 25.1 |
| 200 ppm by wt. | 84.3 | 2.9 | 9.9 | 54.7 | 23.4 | 23.5 |

*)comparative example

1. A process for producing surface postcrosslinked superabsorbent particles, comprising:
polymerizing an aqueous monomer solution or suspension to a polymer gel;
optionally comminuting the polymer gel;
drying the polymer gel;
optionally grinding and classifying the dried polymer gel;
thermally surface postcrosslinking and cooling the dried polymer gel; and
coating resultant polymer particles with at least one pyrazole before, during, and/or after the thermal surface postcrosslinking;
wherein the aqueous monomer solution or suspension comprises:
a) at least one ethylenically unsaturated carboxylic acid that has been at least partly neutralized,
b) at least one crosslinker, and
c) at least one initiator.

2. The process according to claim 1, wherein the pyrazole comprises at least one selected from the group consisting of:
a compound of general formula (I): 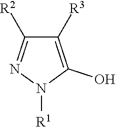
where;
R¹ is C₁- or C₂-alkyl,
R² is H, C₁- to C₂₀-alkyl or C₆- to C₂₀-arylalkyl; and
R³ is H, C₁- to C₂₀-alkyl or C₆- to C₂₀-arylalkyl;
a compound of general formula (II): 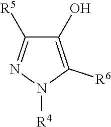
where:
R⁴ is C₁- or C₂-alkyl,
R⁵ is H, C₁- to C₂₀-alkyl or C₆- to C₂₀-arylalkyl, and
R⁶ is H, C₁- to C₂₀-alkyl or C₆- to C₂₀-arylalkyl; and
a compound of general formula (III); 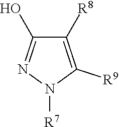
where:
R⁷ is C₁- or C₂-alkyl,
R⁸ is H, C₁- to C₂₀-alkyl or C₆- to C₂₀-arylalkyl, and
R⁹ is H, C₁- to C₂₀-alkyl or C₆- to C₂₀-arylalkyl.

3. The process according to claim 1, wherein the ethylenically unsaturated carboxylic acid comprises acrylic acid.

4. The process according to claim 1, wherein the initiator comprises a peroxodisulfate.

5. The process according to claim 1, wherein the polymer particles are coated with 0.001% to 1% by weight of the pyrazole, based on a total weight of the polymer particles.

6. The process according to claim 1, wherein the polymer particles are coated with 0.01% to 0.1% by weight of the pyrazole, based on a total weight of the polymer particles.

7. The process according to claim 2, wherein:
in general formula (I), R¹ is C₁-alkyl, R² is C₁- to C₃-alkyl or C₆- to C₈-arylalkyl, and R³ is H, C₁- to C₃-alkyl or C₆- to C₈-arylalkyl;
in general formula (II), R⁴ is C₁-alkyl, R⁵ is C₁- to C₃-alkyl or C₆- to C₈-arylalkyl, and R⁶ is C₁- to C₃-alkyl or C₆- to C₈-arylalkyl; and
in general formula (III), R⁷ is C₁-alkyl, R⁸ is H, C₁- to C₃-alkyl or C₆- to C₈-arylalkyl, and R⁹ is C₁- to C₃-alkyl or C₆- to C₈-arylalkyl.

8. The process according to claim 2, wherein:
the compound of general formula (I) comprises 1,3-dimethyl-5-pyrazolone; and
the compound of general formula (II) comprises at least one selected from the group consisting of 1,5-dimethyl-3-ethyl-4-hydroxypyrazole and 1,5-dimethyl-4-hydroxy-3-phenylpyrazole.

9. Superabsorbent particles obtained by the process of claim 1.

10. Superabsorbent particles obtained by the process of claim 2.

11. Superabsorbent particles obtained by the process of claim 5.

12. Superabsorbent particles obtained by the process of claim 6.

13. Superabsorbent particles obtained by the process of claim 7.

14. Superabsorbent particles obtained by the process of claim 8.

15. A hygiene article comprising superabsorbent particles according to claim 1.
